(19) United States
(12) Patent Application Publication
(10) Pub. No.: US 2019/0261918 A1
(43) Pub. Date: Aug. 29, 2019

(54) BREATHING GAS ANALYZER FOR ANALYZING A BREATHING GAS

(71) Applicant: Loewenstein Medical Technology S.A., Luxembourg (LU)

(72) Inventor: Matthias SCHWAIBOLD, Karlsruhe (DE)

(21) Appl. No.: 16/285,405

(22) Filed: Feb. 26, 2019

(30) Foreign Application Priority Data
Feb. 28, 2018 (DE) 102018001557.3

(51) Int. Cl. A61B 5/00; A61B 5/08; A61M 16/00; A61M 16/06; A61M 16/08

(52) U.S. Cl. CPC A61B 5/4815; A61B 5/08; A61B 5/4812; A61M 16/08; A61M 16/024; A61M 16/0683; A61B 5/7278

(57) ABSTRACT

The invention relates to a respiratory gas analyzer for analyzing a respiratory gas, which is configured to determine at least one sleep stage from the respiratory gas and to determine a sleep quality based on the sleep stage. Furthermore, the invention relates to a respirator which comprises such a respiratory gas analyzer, to a device for carrying out a polygraph and/or polysomnograph, which comprises such a respiratory gas analyzer, and to a method for controlling a respirator which comprises such a respiratory gas analyzer.

### CROSS-REFERENCE TO RELATED APPLICATIONS

The present application claims priority under 35 U.S.C. § 119 of German Patent Application No. 10 2018 001 557.3, filed Feb. 28, 2018, the entire disclosure of which is expressly incorporated by reference herein.

### BACKGROUND OF THE INVENTION

### 1. Field of the Invention

The present invention relates to a respiratory gas analyzer for analyzing a respiratory gas.

### 2. Discussion of Background Information

Many people suffer from poor sleep, which results in decreasing concentration and reduced intellectual performance. Poor sleep can also be responsible for disturbances of other functions, such as for example, muscle tension, respiration, heart rate, blood pressure, body temperature, hormones, and metabolism. The causes of poor sleep can be manifold in this case. Poor sleep often results from a sleep disturbance of the patient. Poor sleep can also exist, however, although no sleep disturbance has been able to be determined.

One possible cause of a sleep disturbance can be an irregularity in the respiration (respiratory disturbances). The most frequent sleep disturbance is obstructive sleep apnea (OSA); in this case, the respiratory flow is temporarily either substantially reduced (hypopnea) or completely interrupted (apnea). The lack of oxygen occurring as a result generates a stress situation, which suppresses restful sleep and damages the heart in the medium term.

To be able to make a statement about the extent of a sleep disturbance of a patient, to be able to treat and/or remedy it accordingly, it is necessary to carry out a sleep diagnosis.

A sleep diagnosis can be performed, for example, by polygraph devices or respirators.

The polygraph devices are configured to carry out a polygraph. The polygraph is a method which is predominantly used in home diagnostics. In this case, the polygraph device/measuring device configured for the polygraph is sent home with the patient. The polygraph device/measuring device records items of information about parameters such as, for example, a nocturnal oxygen saturation, a pulse rate, a position of the body of the patient during sleep, and about respiration of the patient in sleep, including snoring. Depending on the type of the polygraph device/measuring device, the polygraph device/measuring device can comprise an electrocardiogram (ECG) or electromyogram (EMG), which can detect a nocturnal heart activity or a leg muscle activity, respectively, of the patient in sleep. The detected parameters can be used for a statement about the extent of the sleep disturbance. Furthermore, the function of the respirators and/or the respiratory therapy can be monitored via the detected parameters.

Polygraph devices are often used together with respirators. The respirators used in polygraphs are configured in this case to carry out the standard therapies, in particular CPAP therapy (continuous positive airway pressure), APAP therapy (automatic positive airway pressure), and bi-level therapy. Some of the described respirators or therapies, in particular APAP therapy and/or bilevel therapy can also be configured to detect respiratory gas parameters in order to determine, for example, a required respiratory gas pressure of a patient from breath to breath and to supply the patient accordingly. For this purpose, the respirators generally comprise at least one pressure sensor and/or one flow sensor for determining a respiratory gas pressure or a respiratory gas volume, respectively.

The previously known polygraph devices and respirators can thus be configured to detect respiratory gas parameters and to determine an extent of a sleep disturbance which is induced by sleep-related respiratory disturbances. However, they cannot determine sleep disturbances which are not related to respiratory disturbance. Therefore, the previously known polygraph devices and respirators cannot conclude a sleep quality (poor or good sleep) of the patient.

To also be able to make a statement about the sleep quality of the patient in sleep diagnostics, in addition to the extent of the sleep disturbance, it is presently necessary to carry out a polysomnograph in a sleep laboratory. In the polysomnograph, items of information/parameters about the current sleep state and about the sleep quality of the patient are continuously detected by means of a complex measurement set up in a sleep laboratory.

The monitored polysomnograph in sleep medicine has been considered up to this point to be a basic instrument and a reference method in the instrumental diagnostics of sleep disturbances. The polysomnograph comprises in this case, inter alia, the recordings of sleep EEG (brain current picture), EOG (eye-movement), EMG (muscle tension), ECG (cardiac rhythm), respiratory flow, breathing effort, oxygen saturation, body temperature, body position, and leg movement. Before carrying out a polysomnograph in the sleep laboratory, the patient is generally observed for a day to recognize his activities and habits. Normally the patient subsequently spends two nights in the sleep laboratory.

The detection of the sleep quality by means of the polysomnograph has the disadvantage that the polysomnograph device is very costly due to the complex construction. The method of the polysomnograph is very time-consuming, since the patient has to be admitted as an inpatient for multiple days in the sleep laboratory. A home application by the patient is thus not possible. Moreover, an analysis of the obtained items of information/parameters can only take place on the next day.

It would therefore be advantageous to provide a device which is configured to provide a patient and/or attending technician, in addition to a statement about an extent of a sleep disturbance (sleep-related respiratory disturbance) of the patient, also a simplified and direct statement about their sleep quality.

### SUMMARY OF THE INVENTION

According to the invention, the respiratory gas analyzer is configured to determine at least one sleep stage from the respiratory gas and to determine a sleep quality based on the sleep stage. For the determination of the sleep quality, the respiratory gas analyzer generally comprises at least one preprocessing unit, at least one buffer memory unit, at least one detector unit, and at least one integrator unit. The at least one preprocessing unit, the at least one buffer memory unit, the at least one detector unit, and the at least one integrator unit are configured to exchange data with one another. The respiratory gas analyzer can optionally also comprise a measuring unit for detecting respiratory gas parameters and/or an evaluator unit for evaluating the determined sleep quality. The preprocessing unit is generally configured to acquire at least one respiratory gas parameter from a measuring system, wherein the measuring system is generally arranged externally to the respiratory gas analyzer, for example, in a respirator or on a patient. The at least one integrator unit can be designed as integrated into the respiratory gas analyzer or can be arranged as an external unit, for example, in a respirator.

The respiratory gas analyzer according to the invention can be used, for example, in a respirator and/or a device for carrying out a polygraph and/or a polysomnograph. In this case, the respiratory gas analyzer is usable both in home respiration and also in clinical respiration. This offers the advantage that, for example, the field of use of respirators which comprise a respiratory gas analyzer according to the invention is expanded. A compact respirator can thus be provided to a patient, which also takes over the monitoring of the sleep quality. The necessity of carrying out an EEG can thus be significantly reduced. A respirator configured in this way offers better monitoring of the success of a respiratory therapy in the case of therapy-accompanying application by a patient and/or attending technician.

In one embodiment, the respiratory gas analyzer is configured to render the sleep quality as a sleep quality index. The sleep quality index is used to represent a determined sleep quality in a user-friendly manner, so that a patient can read off a statement about the sleep quality without medical technical knowledge. The respiratory gas analyzer is configured to represent the sleep quality as a sleep quality index, wherein a low sleep quality index indicates worse sleep and a higher sleep quality index indicates better sleep. In general, the sleep quality index is displayed as a numeric value between 0 and 1 or in the form of a prepared graphic, for example, a curve diagram or pie chart. The sleep quality index offers the possibility of analyzing and/or pre-filtering complex respiratory gas parameters for the patient and/or the technician and to provide simplified feedback with respect to the sleep stages and/or the sleep quality of the patient over a specific time period. The patient thus receives direct feedback, which is comprehensible to the patient, about the sleep quality of the last night.

The respiratory gas analyzer is thus configured to analyze complex respiratory gas parameters of the respiratory gas by way of the sleep quality index and to provide the patient or technician with their sleep quality as a numeric value or simplified graphic. Upon a representation of a worse sleep quality by a low sleep quality index, in addition the detected respiratory gas parameters can be accessed in a targeted manner, for example, by a technician/caregiver, to obtain detailed items of information about the course of the respiratory gas parameters over a time period, for example, an entire night. The patient can also seek out a physician, for example, in the event of repeatedly occurring poor sleep.

In a further embodiment of the invention, the respiratory gas analyzer is configured to determine the at least one sleep stage from at least one respiratory gas parameter of the respiratory gas, wherein the respiratory gas analyzer is configured to acquire the at least one respiratory gas parameter from a measuring system. The measuring system generally comprises at least one sensor, wherein the sensor is typically a pressure sensor. The respiratory gas analyzer and/or the measuring system can optionally comprise further sensors for measuring further respiratory gas parameters, for example, a flow sensor. The respiratory gas pressure and/or the respiratory gas volume per breath is typically determined. The measuring system is preferably arranged externally to the respiratory gas analyzer, for example, on the patient.

In an alternative refinement, the respiratory gas analyzer is configured to determine the at least one sleep stage from at least one respiratory gas parameter of the respiratory gas, wherein the respiratory gas analyzer comprises at least one measuring unit, which is configured to determine the at least one respiratory gas parameter from the respiratory gas. The measuring unit can comprise at least one pressure sensor and/or at least one flow sensor, wherein the at least one pressure sensor and/or the at least one flow sensor is configured to enable a continuous measurement of respiratory gas parameters and thus continuous monitoring of the respiratory function of the patient. The pressure sensor can detect a respiratory gas pressure, for example. The flow sensor generally detects a respiratory gas volume. Preferably, the respiratory gas pressure and/or the respiratory gas volume per breath is determined.

In one refinement, the respiratory gas analyzer comprises at least one preprocessing unit, which is configured to analyze the determined or provided respiratory gas parameter and to determine at least one coefficient and/or at least one respiratory gas signal. A respiratory gas signal is in this case generally the sum of at least two coefficients over a predetermined time period. A predetermined time period can be one breath, for example. The at least one respiratory gas signal can be, for example, a respiratory gas volume of a current breath or a current breathing rate. Possible further respiratory gas parameters for determining the at least one coefficient and/or the at least one respiratory gas signal can be a respiratory gas flow, a respiratory gas contour, an inspiratory or expiratory tidal volume, a breathing rate, a breath duration, an inspiration duration, an expiration duration, a peak flow, or a leakage.

Moreover, possible respiratory gas parameters can each be a change of the above-mentioned respiratory gas parameters instead of the absolute values thereof. Possible respiratory gas parameters can also each be a variation of the mentioned respiratory gas parameters instead of the absolute values thereof, for example, a deviation from a mean value, a deviation from a smoothed value, a standard deviation, or a measure of variation from chaos theory. Furthermore, possible respiratory gas parameters can each be a variation of the frequency spectrum of the above-mentioned respiratory gas parameters instead of the observation in the time range, for example, an expression of at least one peak in accordance with a breathing rate. For example, the respiratory gas parameter can be a respiratory gas volume determined by the measuring system, for example, per breath. The respiratory gas volume supplies items of information about the quantity of the respiratory gas which is inhaled during an inspiration and is exhaled during an expiration. The respiratory gas volume thus enables a statement about a depth of an inspiration or an expiration of the patient. In an adult, a respiratory gas volume is generally 4-7 ml per kilogram of body weight. This corresponds to approximately 4 l of respiratory gas per minute. An inspiration is generally 1.5 seconds long, while an expiration is generally approximately 2 seconds long. By determining the respiratory gas volume over a predetermined time period, a statement can be made about how deeply a patient breathes in the course of the night. In general, the preprocessing unit is configured to determine a respiratory gas volume per breath, wherein a current respiratory gas volume per breath is scaled as a percentage value of a normal value of an inspiratory respiratory minute volume of a specific time, for example, the last ten minutes (normalized breathing volume). The preprocessing unit is thus configured to determine a scaled value, in particular a scaled respiratory gas volume.

In one embodiment, the respiratory gas analyzer comprises at least one buffer memory unit, which is configured to buffer the at least one coefficient and/or the at least one respiratory gas signal and/or the scaled value. The last N coefficients and/or N respiratory gas signals and/or N scaled values, for example, breath volumes per breath or minute, are preferably buffered, wherein N is at least 2. The at least one buffered coefficient and/or the at least one buffered respiratory gas signal and/or the at least one scaled value are generally made available to a detector unit of the respiratory gas analyzer for a predetermined time period, preferably between five seconds and five minutes. The buffer memory unit is particularly preferably configured to store the determined coefficients and/or respiratory gas signals and/or scaled values of a recording period, for example, an entire night.

In a further embodiment, the respiratory gas analyzer comprises at least one detector unit, which is configured to determine at least one sleep stage from the at least one buffered coefficient and/or the at least one buffered respiratory gas signal and/or the at least one scaled value. In general, the detector unit is configured to determine a deflection of the coefficient and/or the buffered respiratory gas signal and/or the scaled/normalized value, for example, of the respiratory minute volume. The detector unit is configured to strongly low-pass filter the determined deflection. The detector unit is typically configured to filter over 2 minutes using a second-order smoothing filter. This offers the advantage that the relevance of individual large deflections is reduced. The detector unit is configured to render such a smoothed value as a respiration disturbance index, which corresponds to low-pass smoothing (deflection). The disturbance index is a reciprocal value of a stability level. The detector unit is configured to associate a current breath with a sleep stage in accordance with the determined value of the disturbance index or the stability level known by way of the disturbance index. For example, at a disturbance index of <1.5, a breath can be associated with a deep sleep, at a disturbance index of 1.5-3, with light sleep, at a disturbance index of 3-5, with REM sleep, and at a disturbance index of >5, a waking state.

The detector unit is optionally configured to perform at least one classification into one of the sleep stages waking phase, REM sleep phase, light sleep phase/NREM 1 or 2, deep sleep phase/SWS or NREM 3 or 4. The detector unit is configured to determine at least one stability level, for example, from the at least one normalized value, for example, a normalized respiratory gas volume. A stability level is to be understood as an instantaneous stability, a current value, of a respiratory gas volume or of a respiratory gas flow. An inverted stability level corresponds in this case to the disturbance index.

The stability level can be determinable, for example, by a comparison of a current value, for example, a respiratory gas volume per minute, to a corresponding normalized value of the respiratory gas volume. With a stable respiratory gas volume, the stability level is generally high, with a varying respiratory gas volume, the stability level is generally low. The detector unit is preferably configured to transmit the determined stability level and/or the disturbance index back to the buffer memory unit for buffering.

The detector unit is alternatively configured to perform a classification into at least one sleep stage based on the at least one determined stability level. For example, the detector unit can be configured to conclude a deep sleep in the case of a stability level above a predetermined threshold value between 0 and 1, for example, at 0.8.

A sleep stage of a patient may optionally also be concluded via the variation of the respiratory gas signal based on the respiratory gas volume or the breathing rate. A classification into a sleep stage via a frequency or periodicity of the respiratory gas signal determined by the preprocessing unit, for example, via a variation of the frequency of the respiratory gas signal, can also be carried out. The association of the determined variation with a sleep stage can be carried out, for example, via an association probability (fuzzy association).

In a further embodiment, the respiratory gas analyzer comprises at least one integrator unit, which is configured to determine a sleep quality based on the at least one sleep stage determined by the detector unit. In general, the integrator unit is configured to add up the time and the proportion of the night in which the disturbance index was in the range for deep sleep, in the range for light sleep, for REM sleep, or the waking state at the end of a measuring period, for example, one night. The integrator unit is configured to determine the sleep quality based on the duration of the detected deep sleep or the ratio of the duration of the deep sleep and the average age.

The integrator unit is preferably configured to render the sleep quality as a sleep quality index. The sleep quality index supplies a statement about how well or poorly a patient has slept. The sleep quality index is preferably rendered as a numeric value between 0 and 1 or between 0% and 100%, alternatively as a deep sleep duration in minutes/hours. The sleep quality index can optionally be rendered as a graphic. For this purpose, the stability levels or sleep stages or associations with sleep stages determined at every point in time by the detector unit are added up over the entire night. For example, all sections having deep sleep are added up, either uniformly weighted or weighted according to the deep sleep probability thereof or the stability level thereof. The sleep quality index can thus, for example, be determinable from an integral of the curve profile of the stability level.

The respiratory gas analyzer preferably comprises at least one evaluator unit, wherein the evaluator unit is configured to compare the determined sleep quality and/or the at least one determined stability level and/or the at least one determined sleep stage to at least one target value and evaluate it. For example, the evaluator unit is configured to compare the determined sleep quality of the patient to age-typical values and evaluate it. Alternatively, a target value can be used in a sex-specific or weight-specific manner for a comparison.

In a refinement, the respiratory gas analyzer is configured to control a polygraph device/diagnostic device and/or a PAP device/respirator based on the at least one determined stability level and/or the at least one determined sleep stage and/or the determined sleep quality.

In one embodiment, the respiratory gas analyzer is configured to transmit the sleep quality index and/or the at least one sleep stage and/or the at least one stability level to an operating and information system and/or to a display of a respirator, wherein the operating and information system or the display is configured to display the sleep quality index and/or the at least one sleep stage and/or the at least one stability level. The respirator generally comprises an operating and information system for its operation. The operating and information system advantageously comprises a display, which is configured to render a sleep quality index and/or the at least one sleep stage and/or the at least one stability level as a numeric value and/or as a graphic. The display can optionally be at least partially formed as a touchscreen.

In a further embodiment, the respiratory gas analyzer comprises an interface, which is configured to interact with a terminal to display the sleep quality on the terminal. In general, the interface comprises a modem to be able to communicate wirelessly with the external terminal. In this case, a terminal can be a tablet, a smart phone, a computer, or another device, which is connected to a network, for example. The interface can be an interface of a respirator or can be connected to an interface of a respirator.

For example, the respiratory gas analyzer can be configured to transmit both the detected respiratory gas parameters and also the determined sleep quality, in general in the form of the sleep quality index, to the terminal and to display them in accordance with the format of the terminal as a numeric value and/or as a graphic, for example, in an appliance, for example, in an application (APP). The sleep quality can optionally be output as a document, for example, in Word format or in PDF format. This offers the advantage that the course or the success of the sleep therapy using the respirator according to the invention can be documented and archived by means of a document output in this manner. This is advantageous in particular in the use by an attending technician.

The subject matter of the invention is also a respirator comprising an above-described respiratory gas analyzer. The respiratory gas analyzer according to the invention is configured for use in a PAP device/respirator and/or in a diagnostic/polygraph device and/or polysomnograph device. A respiratory gas analyzer which is provided for use in a predetermined respirator can be set to requirements of the respirator.

Subject matter of the invention is also a device for carrying out a polygraph and/or a polysomnograph comprising an above-described respiratory gas analyzer. The respiratory gas analyzer according to the invention is configured for use in a PAP device/respirator and/or a diagnostic/polygraph device and/or polysomnograph device. For example, the respiratory gas analyzer according to the invention is usable in home respiration and/or in clinical respiration. This offers the advantage that a respiratory gas analyzer which is provided for use in a predetermined device for carrying out a polygraph and/or a polysomnograph can be set to requirements of the device for carrying out a polygraph and/or a polysomnograph.

Subject matter of the invention is also a method for controlling a respiratory gas analyzer according to one of the above-described features.

In a first step, at least one respiratory gas parameter is acquired by the respiratory gas analyzer from a measuring system. The measuring system can be arranged externally or internally to the respiratory gas analyzer and can comprise at least one sensor. The sensor can be embodied, for example, as a carbon dioxide, oxygen, gas, narcosis gas, respiratory gas, flow sensor or pressure sensor. For example, the respiratory gas analyzer acquires a respiratory gas volume determined by the at least one sensor, for example, a respiratory gas flow sensor.

In a further step, the at least one respiratory gas parameter is preprocessed to form at least one coefficient and/or to form at least one respiratory gas signal and/or a scaled value. In this case, for example, a volume of a current breath or a current breathing rate is determined. In general, a respiratory gas volume per breath is determined, wherein a current respiratory gas volume per breath is scaled as a percentage value of a normal value of an inspiratory respiratory minute volume of a specific time, for example, the last ten minutes (normalized breathing volume).

In a further step, the at least one coefficient and/or the at least one respiratory gas signal and/or the scaled value is buffered. In general, the at least one coefficient and/or the at least one respiratory gas signal and/or the scaled value are provided to a detector unit over a time period of 5 seconds to 5 minutes.

In a further step, at least one stability level is detected from the at least one coefficient and/or the at least one respiratory gas signal and/or the at least one scaled value. In this case, the stability level can be classified into a waking phase, an REM sleep, a light sleep/NREM 1 or 2 and/or a deep sleep/SWS or NREM 2 or 4. The stability level can be determined by a comparison of a current value, for example, a respiratory gas volume per minute, to a corresponding normalized value of the respiratory gas volume.

In a further step, at least one sleep stage is determined from the at least one determined stability level. The stability level can be determined, for example, by a comparison of a current value, for example, a respiratory gas volume per minute, to a corresponding normalized value of the respiratory gas volume. For example, a deep sleep can be concluded in the case of a stability level above a predetermined threshold value between 0 and 1, for example, at 0.8.

In a further step, a sleep quality is determined from the at least one determined sleep stage. The sleep quality supplies a statement about how well a patient sleeps over a predetermined time period, for example, an entire night.

In a further step of the method, the at least one determined sleep quality is displayed as a sleep quality index. The sleep quality index is a simplified representation of the determined sleep quality. The sleep quality index can be displayed in this case as a numeric value and/or as a graphic. The sleep quality index is preferably rendered as a numeric value between 0 and 1 or between 0% and 100%, alternatively as a deep sleep duration in minutes/hours.

In one embodiment of the method according to the invention, the respiratory gas analyzer transmits the determined sleep quality index and/or the at least one determined sleep stage and/or the determined stability level to an evaluator unit for evaluation. The evaluator unit analyzes the determined sleep quality index and/or the at least one determined sleep stage and compares it, for example, to age-typical stored values. The evaluator unit can optionally compare the determined sleep quality index or the at least one determined sleep stage to alternative values.

In a further embodiment of the method, the respiratory gas analyzer transmits the determined sleep quality index and/or the determined sleep stage and/or the at least one determined stability level via an interface to a terminal. In general, the terminal displays the determined sleep quality index and/or the at least one determined sleep stage and/or the stability level. In this case, the terminal can display, for example, the determined sleep quality index as a numeric value and/or as a graphic. The determined sleep quality index and/or the determined sleep stage and/or the at least one determined stability level can also be displayed on an operating element of a device which comprises the respiratory gas analyzer, for example, a respirator.

In one refinement of the method, a respirator and/or a polygraph device and/or a polysomnograph device is controlled by the respiratory gas analyzer on the basis of the at least one determined stability level and/or the at least one determined sleep stage and/or the determined sleep quality. The determined sleep quality index and/or the at least one sleep stage and/or the stability level can give feedback, for example, about the setting of a respirator. The settings of the respirator can be adapted based on the determined sleep stage. A determination of settings of the respirator can be carried out, for example, by an evaluator unit, wherein the at least one determined stability level is transmitted to the evaluator unit for evaluation. This also applies to the use of the respiratory gas analyzer in a polygraph device and/or a polysomnograph device.

### BRIEF DESCRIPTION OF THE DRAWINGS

Preferred exemplary embodiments of the invention will be explained in greater detail hereafter on the basis of greatly simplified schematic illustrations. In the figures:

FIG. 1 shows a fundamental construction of a device for respiration,

FIG. 2 shows an exemplary schematic construction of a respiratory gas analyzer according to the invention for determining a sleep quality,

FIG. 3a

and

FIG. 3b show an exemplary construction of a respiratory gas analyzer according to the invention,

FIG. 4 shows an embodiment of a determination of a sleep quality index according to the invention,

FIG. 5 shows a classification of the stability level in sleep stages to determine the sleep quality index according to the invention.

In the figures, the same design elements each have the same reference signs.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

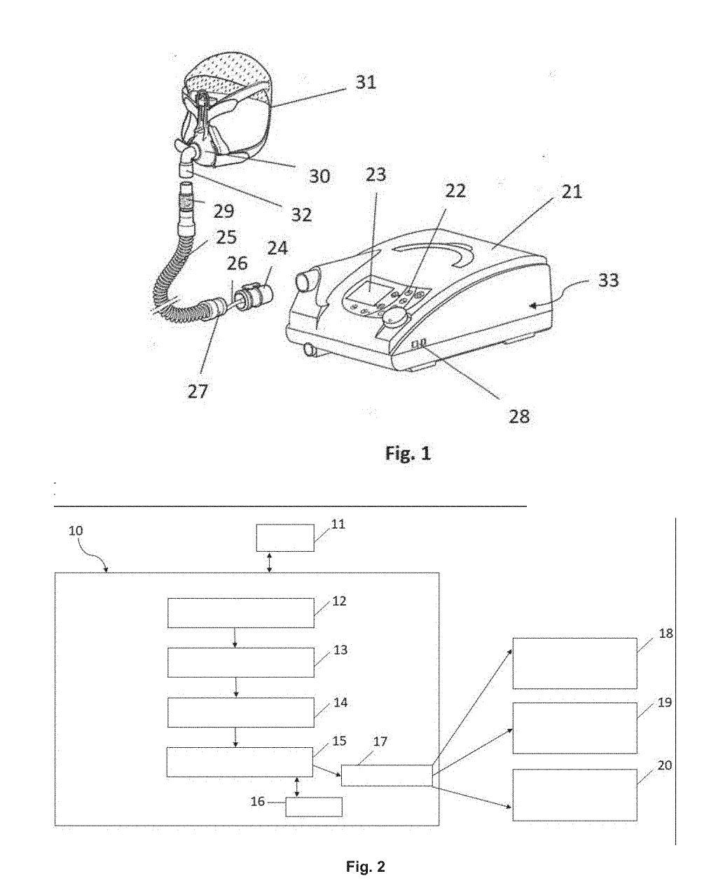

FIG. 1 shows the fundamental construction of a device for respiration. An operating element 22 and an operating and information system 23 consisting of a display which can have a, for example, touch-sensitive, input unit having at least one operating panel, are in the region of a device housing 21 of the respirator 33, having a respiratory gas source in the device interior. A connecting hose 25 is connected via a coupling 24. An additional pressure measuring hose 26, which is connectable via a pressure inlet nozzle 27 to the device housing 21, can extend along the connecting hose 25. The device housing 21 has at least one or also a plurality of interface(s) 28 to enable a data transfer.

A humidifier can moreover be adapted. An exhalation element 29 is arranged in the region of an extension of the connecting hose 25 facing away from the device housing 21. An exhalation valve can also be used.

FIG. 1 additionally shows a patient interface designed as a respiration mask 30, which is implemented as a nasal mask. A fixation in the region of a head of a patient can be performed via headgear 31. The patient interface 30 has a coupling element 32 in the region of its extension facing toward the connecting hose 25.

The input and/or output of data, such as for example, dead space volume, can be performed via the interface 28. The interfaces can be implemented as wired, as an infrared interface, as a Bluetooth interface, or USB. A card slot is preferably also provided. The interface 28 can also be embodied as a LAN interface or as another interface for connection to the Internet. An oxygen supply valve can be adapted to the device for respiration in the region of a device housing. It is conceivable to additionally enrich the respiratory gas with oxygen to improve the patient supply. Instead of one interface 28, a plurality of interfaces can also be provided.

Therapy-external data can also be loaded into the respirator according to the invention and/or executed thereby via the interface 28—for example, embodied as a card slot or USB. The user—when external storage media are recognized by the device—has to confirm a query in the operating panel, whereupon the data are alternately stored or executed in the region of the respirator.

The input and/or output of telemedicine data can take place via the interface 28. For this purpose, for example, mobile wireless or short-range wireless data or WLAN or Bluetooth or network data are received/transmitted via the interface.

The respirator 33 is designed so that it can be connected via a hose and a patient interface 30 to a patient to provide respiration. It comprises a source for respiratory gas, which is designed, for example, as an electric motor having fan wheel, and a unit for determining pressure and/or flow and/or volume of the respiratory gas, and also a control unit, which is designed so that it determines a respiratory gas pressure for each respiration cycle on the basis of a predetermined value for the patient and/or on the basis of measurement signals for the parameters pressure and/or flow and/or volume and regulates the source for respiratory gas in such a way that the respiratory gas pressure is generated.

FIG. 2 shows an exemplary schematic construction of the respiratory gas analyzer 10 according to the invention, which comprises a preprocessing unit 12, a buffer memory 13, an integrator unit 14, and a detector unit 15. The respiratory gas analyzer 10 is arranged in a respirator—shown in FIG. 1. The respiratory gas analyzer 10 can determine at least one sleep stage from the respiratory gas and determine a sleep quality based on the sleep stage.

The respiratory gas analyzer 10 is configured to acquire the at least one respiratory gas parameter from the measuring system 11. For example, a respiratory gas parameter is a respiratory gas volume. The measuring system 11 is arranged externally to the respiratory gas analyzer 10. For example, the measuring system 11 can be arranged in a PAP device/respirator, a diagnostic/polygraph device, a polysomnograph device, or on a patient.

In the present exemplary embodiment, the preprocessing unit 12 is configured, based on the acquired respiratory gas volume, to determine at least one scaled value of the respiratory gas volume, for example, over a time period of 1 minute.

The buffer memory unit 13 is configured to buffer the scaled value and provide it to the detector unit 14. The detector unit 14 acquires the at least one scaled value from the buffer memory and determines at least one stability level based thereon. The stability level can be determined, for example, by a comparison of a current value, for example, the respiratory gas volume per minute, to a corresponding normalized value of the respiratory gas volume. The detector unit is preferably configured to transmit the determined stability level back to the buffer memory unit for buffering. The detector unit is configured to perform a classification into at least one sleep stage based on the at least one determined stability level. For example, the detector unit can be configured to conclude a deep sleep at a stability level above a predetermined threshold value between 0 and 1, for example, at 0.8.

The integrator unit 15 is configured to determine a sleep quality based on the at least one sleep stage determined by the detector unit 14. The sleep quality is rendered as a sleep quality index. The sleep quality index can be indicated on the operating and information system/display 23—shown in FIG. 1—or on the terminal 20 using a scale between 0 and 100 or displayed as a graphic. The scale is adaptable. A lower sleep quality index typically indicates poor sleep, while a higher sleep quality index indicates better sleep. The sleep quality index can be provided to the patient and/or a technician as a numeric value and/or as a graphic.

The respiratory gas analyzer 10 can be configured to transmit the determined sleep quality index and/or at least one determined sleep stage to an evaluator unit 16, to compare it to reference values, for example, for an age, and evaluate it. The evaluator unit 16 can be arranged internally or externally to the respiratory gas analyzer. The evaluator unit 16 transmits the at least one evaluated sleep stage and/or the evaluated sleep quality index back to the integrator unit 15. The respiratory gas analyzer 10 is configured to transmit the at least one evaluated sleep stage or the evaluated sleep quality index to the operating and information system/display 23—shown in FIG. 1—or by means of an interface 17 to the terminal 20.

The interface 17 is thus configured to interact with a terminal 20 to display the sleep quality on the terminal 20. The interface 17 comprises in this case a modem, whereby the respiratory gas analyzer 10 can communicate with at least one terminal 20 in a network. The interface 17 can generally be activated via the operating and information system/display 23. The interface 17 can correspond to the interface 28 shown in FIG. 1. Based on the determined sleep stage and/or the determined sleep quality and/or the determined stability level, the respiratory gas analyzer 10 is configured to control a polygraph device/diagnostic device and/or a PAP device/respirator.

The respiratory gas analyzer 10 shown in FIG. 2 can be used in a respirator 13 described according to FIG. 1. The respirator 13 is configured for use with a respiration mask 10 connected via a respiration hose. The respiration mask 10 and the respiration hose 5 are individually selectable in this case. The respirator 13 can be a respirator of the CPAP class, the APAP class, or the bilevel class. The respiratory gas analyzer 10 according to the invention can optionally be used for a therapy comprising at least three respiration pressure levels. The respiratory gas analyzer 10 according to the invention is also suitable for use in a diagnostic/polygraph device.

Furthermore, FIG. 2 shows an exemplary sequence of the method according to the invention for controlling a respiratory gas analyzer.

In a first step of the method, at least one respiratory gas parameter is acquired by the respiratory gas analyzer 10 from the measuring system 11. For example, the respiratory gas analyzer 10 acquires a respiratory gas parameter determined by a respiratory gas flow sensor of the measuring system 11, for example, a respiratory gas volume per minute.

In a further step, the at least one respiratory gas parameter is preprocessed by the preprocessing unit 12 to form at least one coefficient or to form at least one respiratory gas signal. A respiratory gas parameter is, for example, a volume of the current breath or a current breathing rate. In general, the preprocessing unit 12 determines, for example, a scaled value over a predetermined time period, for example, one minute, from the respiratory gas volume.

In a further step, in general at least the scaled value, but preferably also the at least one coefficient or the at least one respiratory gas signal, is buffered in the buffer memory unit 13. In a further step, the at least one coefficient and/or the at least one respiratory gas signal are provided to the detector unit 14, for example, provided to the detector unit 14 over a time period of 5 seconds to 5 minutes.

In general, the detector unit 14 acquires the at least one scaled value from the buffer memory and determines, based thereon, at least one stability level. The stability level is determined, for example, by a comparison of a current value, for example, the respiratory gas volume per minute, to a corresponding normalized value of the respiratory gas volume. In general, the detector unit transmits the determined stability level back to the buffer memory unit for buffering. The detector unit performs a classification into at least one sleep stage based on the at least one determined stability level.

In a further step, a sleep quality is determined by the integrator unit 15 from the at least one determined sleep stage. For this purpose, the stability levels or sleep stages or associations with sleep stages determined at every point in time by the detector unit are added up over the entire night.

In a further step, the at least one determined sleep quality is displayed as a sleep quality index. The sleep quality index can be determined from an integral of the curve profile of the added-up stability levels. The sleep quality index can be displayed in this case as a numeric value and/or as a graphic. A lower sleep quality index typically indicates poor sleep, while a higher sleep quality index indicates better sleep.

In a further step, the respiratory gas analyzer can transmit the determined sleep quality index and/or the at least one determined sleep stage and/or the at least one stability level to an evaluator unit 16 for evaluation. The evaluator unit 16 analyzes the determined sleep quality index and/or the at least one determined sleep stage and/or the at least one stability level and compares them, for example, to age-typical stored values. The evaluator unit 16 can optionally compare the determined sleep quality index or the at least one determined sleep stage to alternative values. The evaluator unit 16 typically transmits the evaluated sleep quality and/or the at least one evaluated sleep stage and/or the at least one stability level back to the respiratory gas analyzer 10.

In a refinement of the method according to the invention, a respirator and/or a polygraph device 18, 19 and/or a polysomnograph device is controlled by the respiratory gas analyzer 10 on the basis of the at least one determined sleep stage and/or the determined sleep quality. The determined sleep quality index and/or the at least one sleep stage can give feedback, for example, about the setting of the respirator 33—shown in FIG. 1. Based on the determined sleep stage and/or the determined sleep quality index, the respiratory gas analyzer 10 can adapt the settings of the respirator 33—shown in FIG. 1.

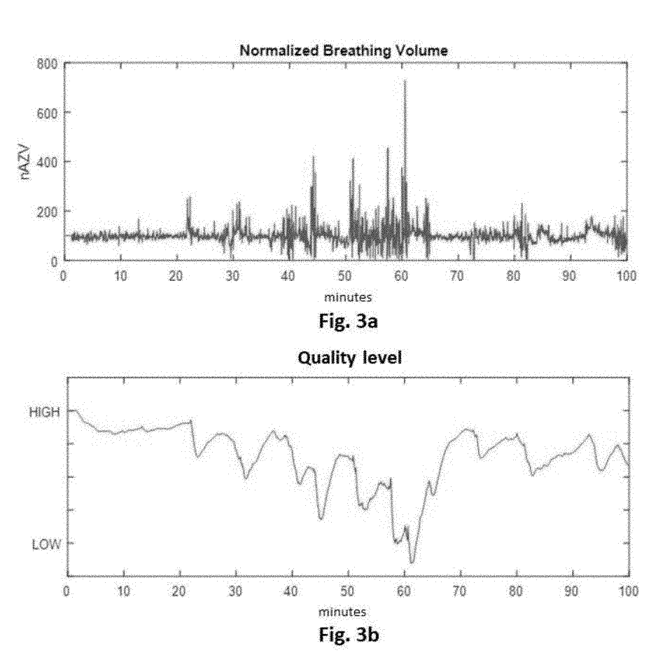

FIG. 3a shows a normalized respiratory gas volume (normalized breathing volume) determined by way of example. In this case, a percentage specification of the normalized respiratory gas volume is plotted on the Y axis and a time span between 0 seconds and 100 minutes is plotted on the X axis. The normalized respiratory gas volume shown in FIG. 3a is determined by a preprocessing unit 12—shown in FIG. 2—having the above-described features.

FIG. 3b shows an exemplary curve of a determined stability level. In this case, the stability level having the classification high and low is plotted on the Y axis and a time span between 0 and 100 minutes is plotted on the X axis. The stability level shown in FIG. 3b is determined by an integrator unit 15—shown in FIG. 2—having the above-described features. With a stable respiratory gas volume, the sleep quality level/quality level is rather high, with a varying respiratory gas volume, it is rather low.

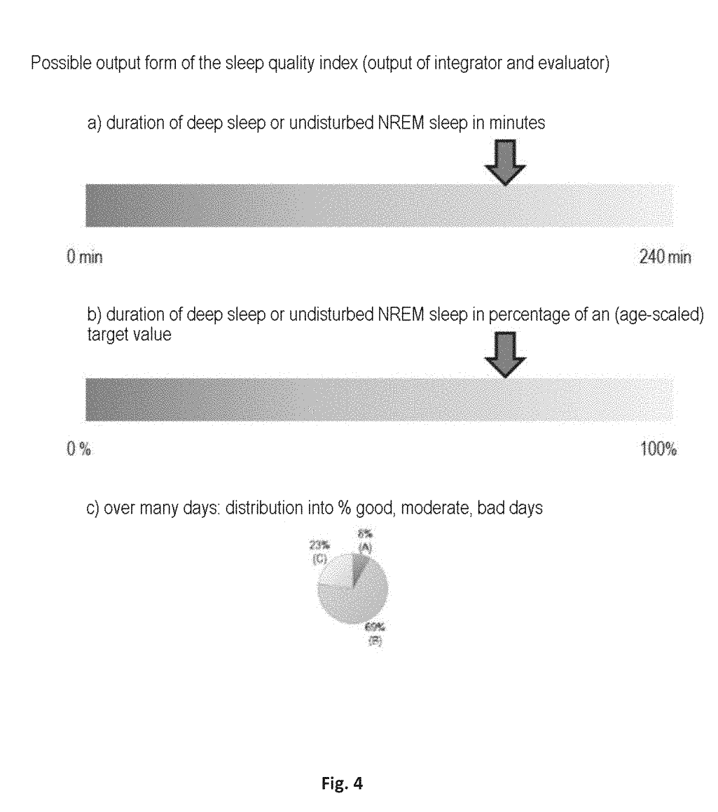

FIG. 4 shows an exemplary illustration of the sleep quality index according to the invention. A scale a) and a scale b) and a pie chart c) are shown. The scale a) is divided into a time period of 0 to 240 minutes. Scale a) shows a duration of a deep sleep phase or an undisturbed NREM sleep in minutes. Scale b) is arranged corresponding to scale a) and is divided in percentage from 0% to 100%. Scale b) shows the duration of the deep sleep phase or the undisturbed NREM sleep in percentage of an age-scaled target value. The pie chart c) shows a representation of distributions over a specific time period in percentage. The pie chart is classified, for example, into good, moderate, and bad days. The distribution/output of the sleep index according to the invention shown in FIG. 4 is by way of example. The output or display of the sleep quality index is adaptable.

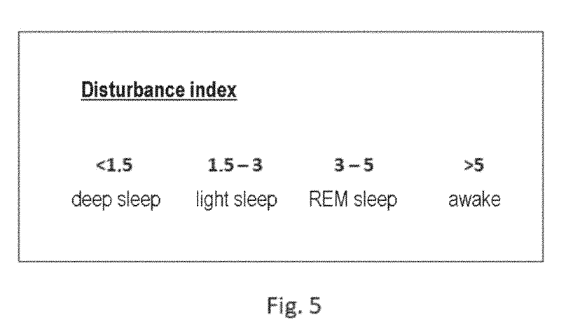

FIG. 5 shows a preferred classification of the stability level into sleep stages to determine the sleep quality index according to the invention.

In a first step of the method—as shown in FIG. 2—a respiratory minute volume in liters/minutes is detected and computed by a measuring unit or the measuring system 11 by the respiratory gas analyzer 10 per breath of a patient. In this case, the inhaled respiratory volume in liters and the duration of the breath in seconds are detected and multiplied by 60. A current respiratory minute volume is thus determined.

In a further step, a mean respiratory minute volume is moreover computed and continuously updated. For example, the mean respiratory minute volume of the last 5 minutes is always detected. Therefore, in addition to the current respiratory minute volume, a mean respiratory minute volume is determined by the respiratory gas analyzer.

In a further step, the determined current respiratory minute volume is scaled into a percentage specification of the mean value. The scaled/normalized respiratory minute volume is thus the respiratory minute volume divided by the mean respiratory minute volume multiplied by 100. For example, a mean respiratory minute volume can be 10 l/min and a current respiratory minute volume can be 11 l/min. The scaled respiratory minute volume is 110% in this example. In a further step, a deflection of the scaled respiratory minute volume is computed. According to the above-mentioned example, a deflection of a scaled respiratory minute volume of 110% is 10. If the current respiratory minute volume corresponds to the mean respiratory minute volume, a deflection of 0 is provided.

In a further step, the deflection is strongly low-pass filtered, for example, over 2 minutes using a second-order smoothing filter. This reduces the relevance of individual large deflections. The smoothed value indicates how distant the deflection was on average from 0 in the last 2 minutes. This is rendered as a breathing disturbance index and corresponds to low-pass smoothing (deflection). The disturbance index is a reciprocal value of the stability level. Depending on the embodiment, a disturbance index (=high value awake, low value deep sleep) or a stability level (inverted) is used as an intermediate result.

In a further step, the current breath is associated with a sleep stage in accordance with the determined value of the disturbance index (which can only change slowly) or of the stability level known by way of the disturbance index. The classification of the disturbance index into the respective sleep stages is shown in FIG. 5. The disturbance index is specified as a numeric value.

At the end of a measurement period, for example, one night, the time and the proportion of the night is added up in the integrator unit in which the disturbance index was in the range for deep sleep, in the range for light sleep, for REM sleep, or the waking state, etc.

In a further step, the sleep quality is generally determined by the duration of the detected deep sleep or the ratio of the duration of the deep sleep and the average age.

### LIST OF REFERENCE SIGNS

10 respiratory gas analyzer

11 measuring system

12 preprocessing unit

13 buffer memory unit

14 detector unit

15 integrator unit

16 evaluator unit

17 interface

18 diagnostic/polygraph device

19 PAP device/respirator

20 terminal

21 device housing

22 operating element

23 operating and information system/display

24 coupling

25 connecting hose

26 pressure measuring hose

27 pressure inlet nozzle

28 interface(s)

29 exhalation element

30 respiration mask

31 headgear

32 coupling element

33 respirator

1.-20. (canceled)

21. A respiratory gas analyzer for analyzing a respiratory gas, wherein the respiratory gas analyzer is configured to determine at least one sleep stage from the respiratory gas and to determine a sleep quality based on the sleep stage.

22. The respiratory gas analyzer of claim 21, wherein the analyzer is configured to render the sleep quality as a sleep quality index.

23. The respiratory gas analyzer of claim 21, wherein the analyzer is configured to determine the at least one sleep stage from at least one respiratory gas parameter of the respiratory gas and to acquire the at least one respiratory gas parameter from a measuring system.

24. The respiratory gas analyzer of claim 21, wherein the analyzer is configured to determine the at least one sleep stage from at least one respiratory gas parameter of the respiratory gas and comprises at least one measuring unit which is configured to determine the at least one respiratory gas parameter from the respiratory gas.

25. The respiratory gas analyzer of claim 21, wherein the analyzer comprises at least one preprocessing unit which is configured to analyze at least one determined or provided respiratory gas parameter and to determine at least one coefficient and/or at least one respiratory gas signal.

26. The respiratory gas analyzer of claim 25, wherein the analyzer further comprises at least one buffer memory unit which is configured to buffer the at least one coefficient and/or the at least one respiratory gas signal.

27. The respiratory gas analyzer of claim 26, wherein the analyzer comprises at least one detector unit which is configured to determine at least one sleep stage from the at least one buffered coefficient and/or the at least one buffered respiratory gas signal.

28. The respiratory gas analyzer of claim 27, wherein the analyzer comprises at least one integrator unit which is configured to determine a sleep quality based on the at least one sleep stage determined by the detector unit.

29. The respiratory gas analyzer of claim 21, wherein the analyzer is configured to control a polygraph device/diagnostic device and/or a PAP device/respirator based on at least one determined stability level and/or at least one determined sleep stage and/or a determined sleep quality.

30. The respiratory gas analyzer of claim 29, wherein the analyzer is configured to transmit the sleep quality index and/or the at least one sleep stage and/or the at least one stability level to an operating and information system and/or to a display of a respirator, the operating and information system or the display being configured to render the sleep quality index.

31. The respiratory gas analyzer of claim 21, wherein the analyzer comprises an interface which is configured to interact with a terminal to display the sleep quality on the terminal.

32. The respiratory gas analyzer of claim 21, wherein the analyzer is configured to determine the at least one sleep stage from at least one respiratory gas parameter of the respiratory gas and to obtain the at least one respiratory gas parameter from a measuring system or a measuring unit which is configured to determine the at least one respiratory gas parameter from the respiratory gas, wherein the analyzer comprises at least one preprocessing unit which is configured to analyze the at least one determined or provided respiratory gas parameter and to determine at least one coefficient and/or at least one respiratory gas signal, and wherein the analyzer comprises at least one detector unit which is configured to determine at least one sleep stage from at least one buffered coefficient and/or at least one buffered respiratory gas signal.

33. The respiratory gas analyzer of claim 21, wherein the analyzer is configured to determine the at least one sleep stage from at least one respiratory gas parameter of the respiratory gas and to obtain the at least one respiratory gas parameter from a measuring system or a measuring unit which is configured to determine the at least one respiratory gas parameter from the respiratory gas, wherein the analyzer comprises at least one preprocessing unit which is configured to analyze the at least one determined or provided respiratory gas parameter and to determine at least one coefficient and/or at least one respiratory gas signal, and wherein the analyzer comprises at least one detector unit which is configured to determine at least one sleep stage from at least one buffered coefficient and/or at least one buffered respiratory gas signal, the analyzer being configured to render the sleep quality as a sleep quality index.

34. The respiratory gas analyzer of claim 21, wherein the analyzer is configured to determine the at least one sleep stage from at least one respiratory gas parameter of the respiratory gas and to obtain the at least one respiratory gas parameter from a measuring system or a measuring unit which is configured to determine the at least one respiratory gas parameter from the respiratory gas, wherein the analyzer comprises at least one preprocessing unit which is configured to analyze the at least one determined or provided respiratory gas parameter and to determine at least one coefficient and/or at least one respiratory gas signal and comprises at least one detector unit which is configured to determine at least one sleep stage from at least one buffered coefficient and/or at least one buffered respiratory gas signal, and wherein the analyzer is configured to render the sleep quality as a sleep quality index and to transmit the sleep quality index and/or at least one sleep stage and/or at least one stability level to an operating and information system and/or to a display of a respirator, the operating and information system or the display being configured to display the sleep quality index.

35. A respirator which comprises the respiratory gas analyzer of claim 21.

36. A device for carrying out a polygraph and/or a polysomnograph , wherein the device comprises the respiratory gas analyzer of claim 21.

37. A method for controlling the respiratory gas analyzer of claim 21, wherein the method comprises:
(a) acquiring of at least one respiratory gas parameter by the respiratory gas analyzer from a measuring system,
(b) preprocessing of the at least one respiratory gas parameter to form at least one coefficient and/or at least one respiratory gas signal and/or at least one scaled value,
(c) buffering the at least one coefficient and/or the at least one respiratory gas signal and/or the scaled value,
(d) detecting at least one stability level of a patient from the at least one coefficient or the at least one respiratory gas signal and/or the scaled value,
(e) determining at least one sleep stage from the at least one stability level,
(f) determining a sleep quality from the at least one determined sleep stage, and
(g) displaying the sleep quality as a sleep quality index.

38. The method of claim 37, wherein the respiratory gas analyzer transmits a determined sleep quality index to an evaluator for evaluation.

39. The method of claim 37, wherein the respiratory gas analyzer transmits the determined sleep quality index and/or the at least one determined sleep stage and/or the at least one determined stability level via an interface to a terminal.

40. The method of claim 37, wherein a respirator and/or a polygraph device and/or a polysomnograph device is controlled by the respiratory gas analyzer on the basis of the at least one determined stability level and/or the at least one determined sleep stage and/or the determined sleep quality.
